(19) United States
(12) Patent Application Publication
(10) Pub. No.: US 2006/0238195 A1
(43) Pub. Date: Oct. 26, 2006

(54) Efficient mapping of reconstruction algorithms for magnetic resonance imaging onto a reconfigurable reconstruction system

(76) Inventors: Ingmar Graesslin, Bonningstedt (DE); Holger Eggers, Schuctzenstrasse (DE)

Correspondence Address: PHILIPS INTELLECTUAL PROPERTY & STANDARDS, 595 MINER ROAD, CLEVELAND, OH, 44143, US

(21) Appl. No.: 10/565,289

(22) PCT Filed: Jul. 16, 2004
(86) PCT No.: PCT/IB04/02331

§ 371 Date: Jan. 20, 2006

Related U.S. Application Data
(60) Provisional application No. 60/489,429, filed on Jul. 23, 2003.

(51) Int. Cl. G01V 3/00; A61B 5/05

(52) U.S. Cl. 324/309; 600/410

(57) ABSTRACT

A magnetic resonance (MR) system (10) includes radiofrequency (R) transmitters (34) which send RF pulses into an examination region (14) to excite a spin system to be imaged. Coil elements (20, 24, 28) pick up an MR signal, which is demodulated and converted into digital data by RF receivers (36). A plurality of independent parallel processing channels (42₁, 42₂, . . . , 42ₐ) is operatively connected to the RF receivers to reconstruct images from the digital data. The parallel processing channels (42₁, 42₂, . . . , 42ₙ) include one or more pipeline stages (54₁, 54₂, . . . , 54ₘ). Processing channels and pipeline stages include a plurality of processing or reconstruction units (52). Processing tasks are dynamically allocated to these processing or reconstruction units on a per scan basis using a single general strategy for mapping processing tasks to hardware resources. The connections (56) between the processing or reconstruction units (52) are reconfigured using a switching means (60). In this manner, different numbers of coil elements (20, 24, 28) can be connected with matching numbers of processing channels (42₁, 42₂, . . . , 42ₙ) to exploit available processing resources optimally.

The present invention relates to diagnostic medical imaging. It finds particular application in conjunction with the reconstruction of magnetic resonance images and will be described with particular reference thereto.

Heretofore, magnetic resonance imaging scanners have included a main magnet, typically superconducting, which generates a temporally constant magnetic field B₀ through an examination region. A radio frequency coil, such as a whole-body coil, and a transmitter tuned to the resonance frequency of the dipoles to be imaged in the B₀ field have often been used to excite and manipulate these dipoles. Spatial information has been encoded by driving the gradient coils with currents to create magnetic field gradients in addition to the B₀ field across the examination region in various directions. Magnetic resonance signals have been acquired by the same coil, demodulated, filtered and sampled by an RF receiver and finally reconstructed into an image on some dedicated or general-purpose hardware.

Rather than using the same coil to transmit and receive RF pulses, the use of surface or local receive coils has become more and more common recently. These receive coils are often arranged in arrays, in which each coil element produces its own output. Instead of combining the outputs of the coil elements in the analog domain, it has proven advantageous to reconstruct the output from individual coil elements separately. Therefore, each coil element is typically connected with its own RF receiver.

While current scanners claim to have a few receive channels with independent RF receivers, they still have only a single reconstruction unit. The processing of the data from each of the RF receivers is interleaved in time in the reconstruction unit, although it may be performed in parallel to reduce reconstruction times.

Simply multiplying the reconstruction units gives rise to the problem of how to map the processing efficiently onto the individual units. A fixed allocation of reconstruction units to receive channels, for example, makes only poor use of available hardware since varying numbers of coil elements might be employed in practice. Moreover, the complexity of the reconstruction software generally increases considerably to divide the processing suitably among the reconstruction units.

The present invention provides an improved imaging apparatus and an improved method, which overcome the above-referenced problems and others.

In accordance with one aspect of the present invention, an MRI system is disclosed. A means creates and transmits RF pulses into an examination region to excite and manipulate a spin system to be imaged. A means picks up an MR signal emitted from the examination region. A means demodulates the MR signal and converts the demodulated MR signal into digital data. A means, including a plurality of reconfigurable processing units with dynamically reconfigurable connections, reconstructs the digital data into images.

In accordance with another aspect of the present invention, a method for processing an MR signal is disclosed. RF pulses are created and transmitted into an examination region to excite and manipulate a spin system to be imaged. The MR signal, emitted from the examination region, is picked up. The picked up MR signal is demodulated and converted into digital data. The digital data is reconstructed into images via a plurality of processing units with dynamically reconfigurable connections.

Advantages of the present invention reside, inter alia, in an increased reconstruction speed due to a more efficient utilization of hardware resources, and simpler reconstruction software architecture due to a single general strategy for mapping processing tasks to hardware resources.

The invention may take form in various components and arrangements of components, and in various steps and arrangements of steps. The drawings are only for purposes of illustrating the preferred embodiments and are not be construed as limiting the invention.

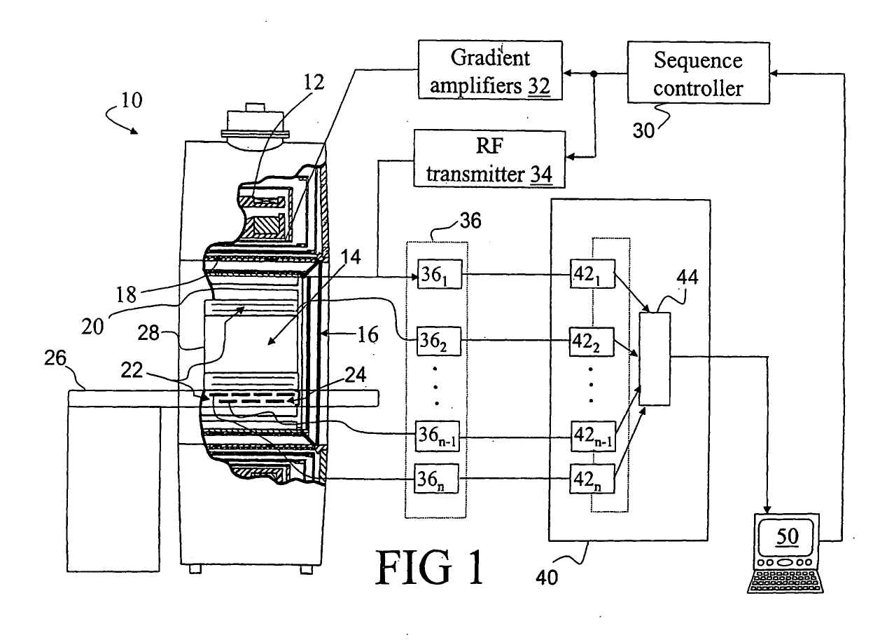

FIG. 1 is a diagrammatic illustration of a magnetic resonance imaging system in accordance with the present invention;

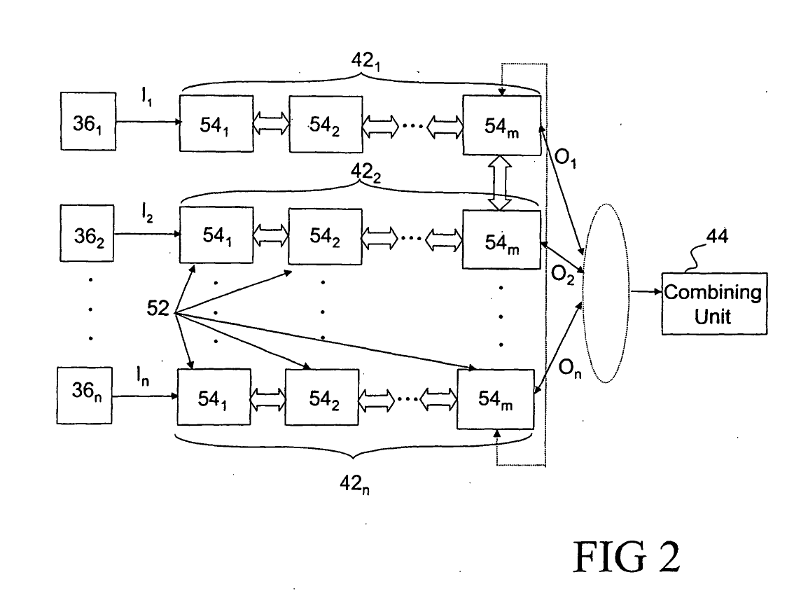

FIG. 2 is a diagrammatic illustration of a reconfigurable reconstruction system in accordance with the present invention;

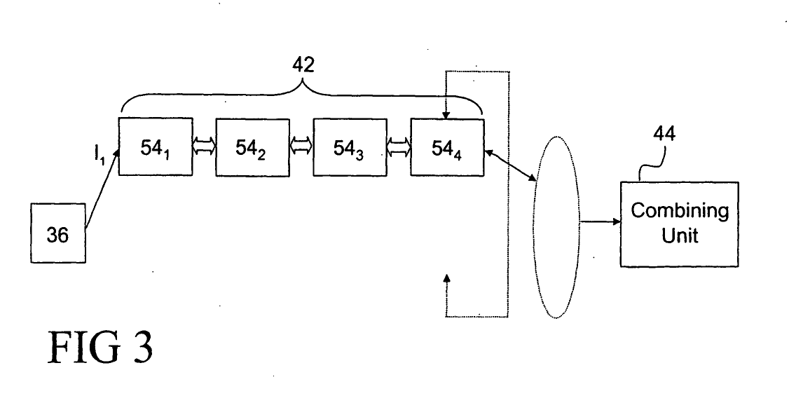

FIG. 3 is a diagrammatic illustration of a possible distribution of processing tasks over four pipeline stages in accordance with the present invention;

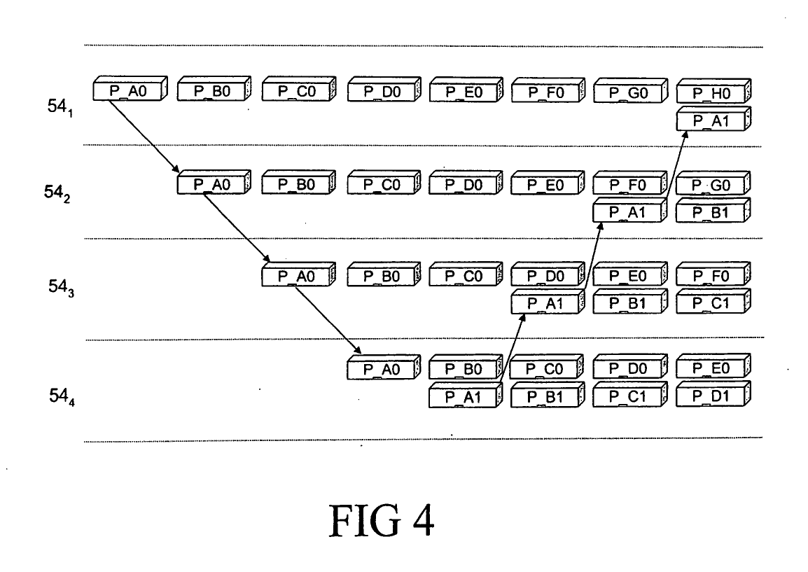

FIG. 4 is a diagrammatic illustration of a possible timing for executing an iterative reconstruction on four processing units per channel in accordance with the present invention;

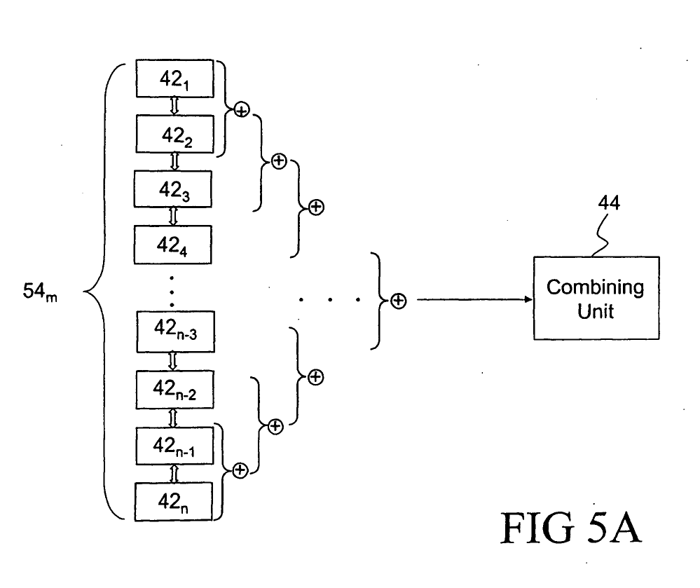

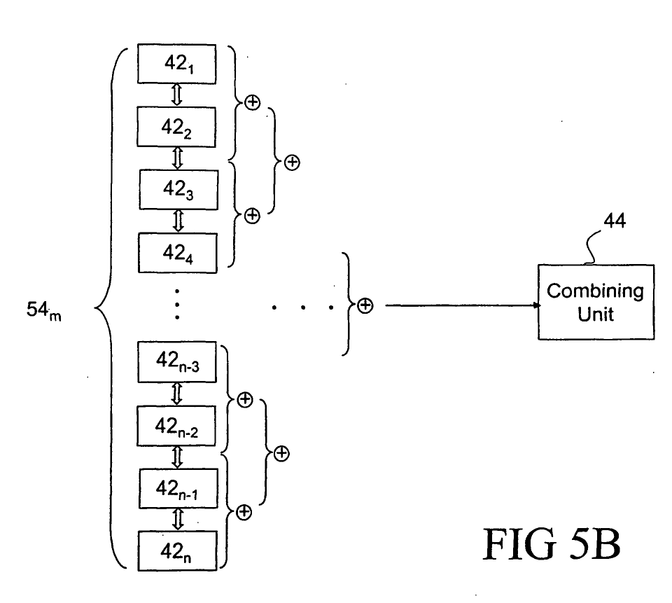

FIGS. 5A-B depict two alternative techniques for combining images from individual processing channels to create a final combined image in accordance with the present invention;

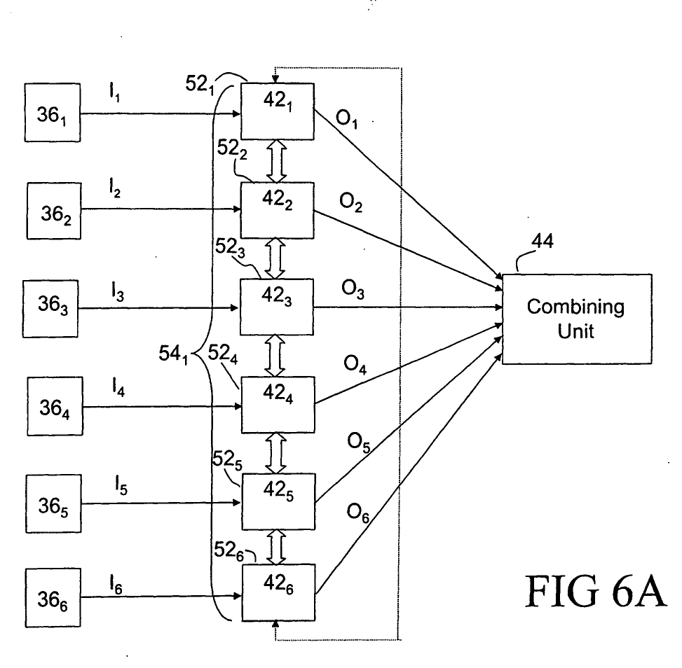

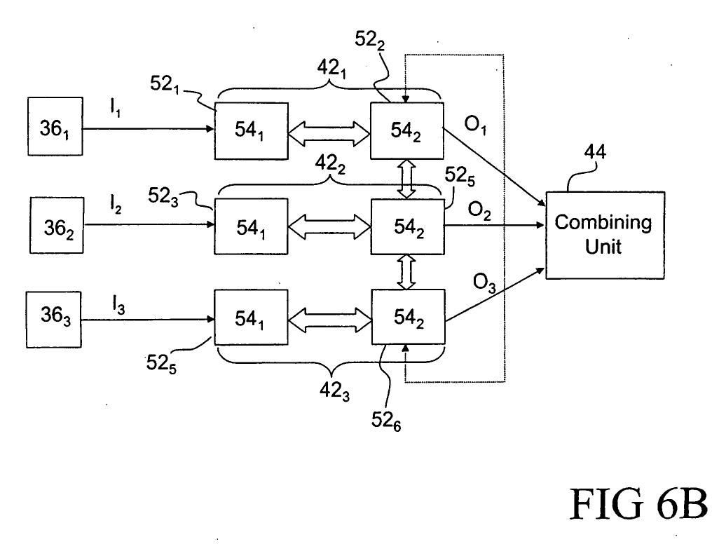

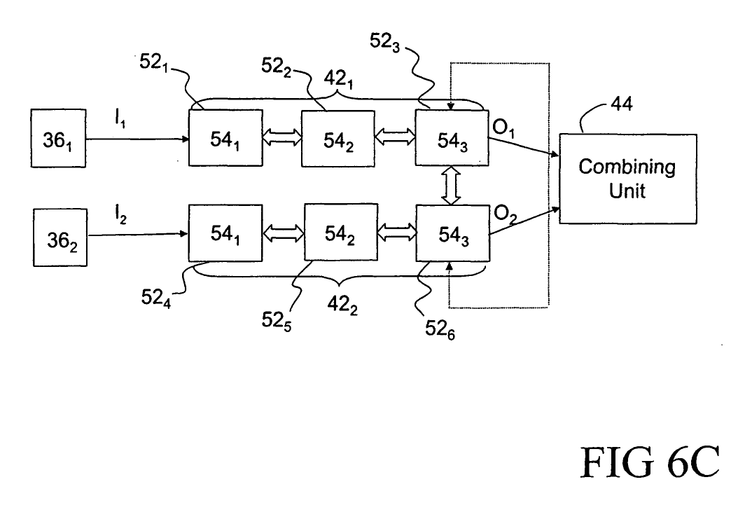

FIG. 6A is a diagrammatic illustration of a reconfigurable reconstruction system utilizing six processing channels with one pipeline stage each in accordance with the present invention;

FIG. 6B is a diagrammatic illustration of a reconfigurable reconstruction system utilizing three processing channels with two pipeline stages in accordance with the present invention;

FIG. 6C is a diagrammatic illustration of a reconfigurable reconstruction system utilizing two processing channels with three pipeline stages each in accordance with the present invention;

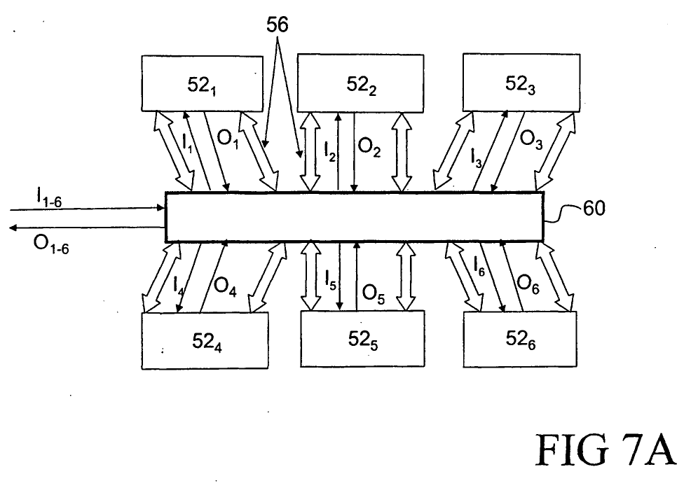

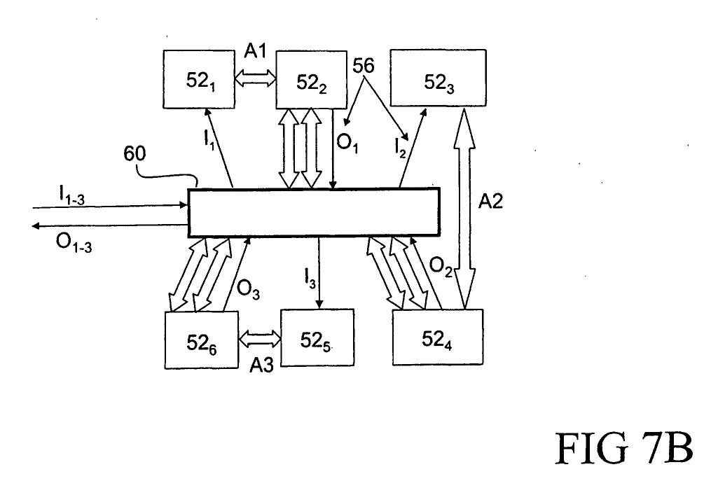

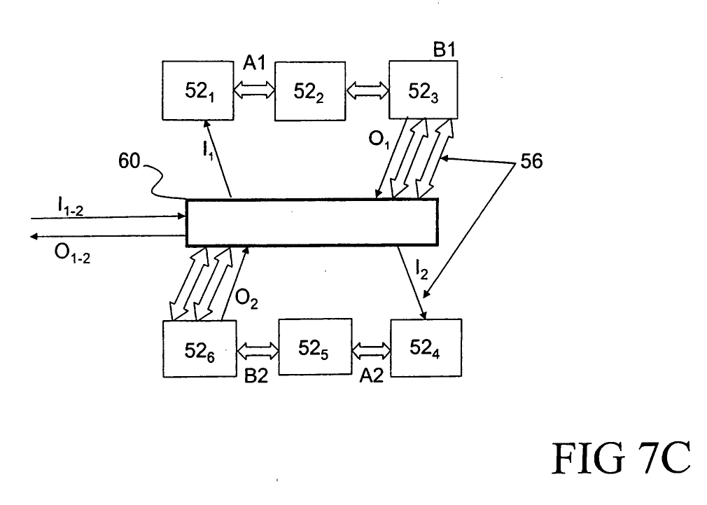

FIGS. 7A-C are diagrammatic illustrations of a reconfigurable reconstruction system built up of boards comprising six embedded processing units each that supports different numbers of processing channels and pipeline stages while utilizing the same total number of processing units, in accordance with the present invention;

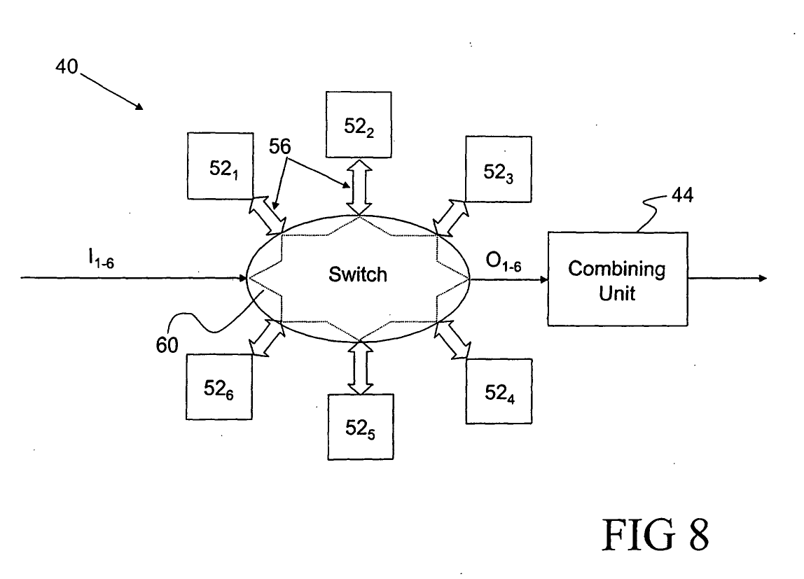

FIG. 8 is a diagrammatic illustration of a reconfigurable reconstruction system built up of a general-purpose hardware, including personal computers or workstations as processing units and a switch as an interconnection.

With reference to FIG. 1, a magnetic resonance (MR) imaging scanner 10 includes a preferably superconducting main magnet 12, which includes a solenoid coil in the illustrated embodiment. The main magnet 12 generates a spatially and temporally constant magnetic field B₀ through an examination region 14 in a bore 16 of the magnet 12.

Magnetic field gradients across the examination region 14 are generated by gradient coils 18 to spatially encode an MR signal, to spoil the magnetization, and the like. In the preferred embodiment, the gradient coils 18 produce gradients in three orthogonal directions, including a longitudinal or z-direction and transverse or x- and y-directions.

A whole-body coil 20, preferably a birdcage coil, transmits radiofrequency (RF) signals for exciting and manipulating a spin system to be imaged and may also receive the MR signal.

A plurality of local RF coils 22 is disposed in the bore 16. The local coils 22 include in the illustrated embodiment a phased-array coil 24, which includes seven coil elements. Optionally, the phased-array coil may be built into a patient support 26. In addition, a surface coil array 28 is disposed in the bore 16. It may include a plurality of surface coils, coils which view different regions of the subject, coils which view a common region of the subject, but have different reception properties, and the like.

To perform measurements, a subject is placed in the magnet's bore 16 with the region of interest in the examination region at or near the magnet's isocenter. A sequence controller 30 controls the gradient amplifiers 32, which drive the gradient coils to create gradient magnetic fields with appropriate strength, orientation and timing. The sequence controller 30 also controls the radiofrequency transmitter 34 which, with the help of the whole-body coil 20, sends radiofrequency pulses into the examination region 14 to excite and manipulate the spin system to be imaged.

Magnetic resonance signals are induced in selected receive coils in the examination region 14. Each of n elements of the local coil arrays 22 is connected with one of n RF receivers 36₁, . . . , 36ₙ. The whole-body coil 20 is also preferably connected to one additional RF receiver.

The reconfigurable reconstruction system 40 supports up to n independent processing channels 42₁, . . . , 42ₙ, with each of these channels connected to one of the RF receivers 36₁, . . . , 36ₙ. The images reconstructed separately by the processing channels are finally combined by the combining unit 44. The combined images (and optionally the uncombined images) are sent to the host computer 50 for storage and viewing. The host computer 50, preferably a personal computer or workstation, includes a display and a user interface connected with the sequence controller 30, which allows the operator to select among a variety of sequences and imaging parameters.

With continuing reference to FIG. 1 and further reference to FIG. 2, the data provided by coils 20, 22, 28 are sent via the RF receivers or receive channels 36₁, . . . , 36ₙ to corresponding individual channels of a plurality of processing channels 42₁, 42₂, . . . , 42ₙ. The data are processed by a plurality of processing or reconstruction units 52, arranged in the pipeline stages 54₁, 54₂, . . . 54ₘ. The allocation of processing or reconstruction units 52 to processing channels and pipeline stages is performed dynamically on a per scan basis. Moreover, the number of processing channels is adapted to the number of receive channels actually in use, i.e. it is chosen to be a multiple or a factor of the number of active receive channels, or to be the same. The images reconstructed separately by the processing channels 42₁, 42₂, . . . , 42ₙ are sent to the combining unit 44, where the images are combined.

With reference to FIG. 3, one of the processing channels of the reconstruction system is shown in more detail. The reconstruction is performed using four pipeline stages 54₁, 54₂, 54₃, and 54₄. The first pipeline stage 54₁ operates on the data in k-space. It performs, for instance, a sampling density compensation or a regridding. The intermediate pipeline stages 54₂ and 54₃ transform the data from k-space to spatial (or image) domain. The use of two pipeline stages permits, in this case, to separate the two-dimensional Fourier transform required in two-dimensional imaging into two subsequent one-dimensional Fourier transforms, allocating one of them to each pipeline stage. The final pipeline stage 54₄ operates on the data in the image domain. It performs, for instance, a roll-off correction or weighting. Alternatively, the images from the individual processing channels are also partly or completely combined in the final pipeline stage to drastically reduce the required bandwidth to the combining unit. In case of an iterative reconstruction, for which a variety of algorithms are known, these processing steps make up the forward processing. Keeping the same mapping of processing tasks to the pipeline stages, the backward processing can be implemented similarly by sending the data in reverse direction from the last to the first pipeline stage. In addition, some further processing in the spatial domain has to be implemented in the last pipeline stage. It includes the core of the iterative reconstruction, such as the conjugate gradient or the generalized minimum residual method, but without the matrix-vector multiplication, and a redistribution of the final combined image to all processing or reconstruction units allocated to the last pipeline stage before the beginning of a new iteration.

FIG. 4 shows a possible timing for an iterative reconstruction executed on the four pipeline stages 54₁, 54₂, 54₃, and 54₄ of FIG. 3. P_xy denotes the processing of image x in iteration y. In the initial iteration, an image A is manipulated in pipeline stages 54₁, 54₂, 54₃, and 54₄ using the forward processing. The images B, C, and D enter pipeline stage 54₁ at suitable later times. When the first image A reaches pipeline stage 54₄, pipeline stage 54₁ has processed images B, C, and D in the initial iteration. Then, the backward processing starts with the image A in the first iteration on pipeline stage 54₄. Preferably, a first chain of processors is dedicated to the forward processing and a second chain of processors is dedicated to the backward processing, although the forward and backward processing can also be executed, even simultaneously, on the same processors.

In FIG. 5A and 5B, exemplary techniques for combining images reconstructed separately by the processing channels are shown. The combination is performed by the processing or reconstruction units allocated to the last pipeline stage 54ₘ, which have the capability of exchanging data with each other.

In FIG. 5A, the image from channel 42₁ is combined with the image from channel 42₂, producing an intermediate combined image, which is sent to the adjacent channel 42₃ to be further combined with the image from this channel. At the same time, the image from channel 42ₙ is combined with the image from channel 42ₙ₋₁, producing an intermediate combined image, which is sent to the adjacent channel 42ₙ₋₂ to be further combined with the image from this channel. After the final combined image from all channels 42₁, 42₂, . . . , 42ₙ has been obtained after n/2 steps, it is sent to the combining unit 44 for further processing.

In FIG. 5B, the images from channels 42₁ and 42₂, 42₃ and 42₄, . . . , 42ₙ₋₁ and 42ₙ are combined in parallel. After the final combined image from all channels 42₁, 42₂, . . . , 42ₙ has been obtained, it is sent to the combining unit 44 for further 30 processing. Alternatively, the combination process may be stopped earlier and all remaining intermediate combined images may be sent to the combining unit 44 for further processing.

FIGS. 6A-C illustrate exemplary implementations of the present invention utilizing six processing or reconstruction units 52₁, 52₂, . . . , 52₆. In FIG. 7, six processing or reconstruction units 52₁, 52₂, . . . , 52₆ are configured to process six channels 42₁, 42₂, . . . , 42₆, with a single pipeline stage 54, each. The data from six coil elements are sent to six corresponding processing channels. The six images from each of the processing channels are summed up in the combining unit 44.

In FIG. 6B, six processing or reconstruction units 52₁, 52₂, . . . , 52₆ are configured to process three channels 42₁, 42₂, and 42₃ with two pipeline stages 54₁, 54₂ each. The data from three coil elements are sent to three corresponding processing channels. The three images from each of the processing channels are summed up in the combining unit 44.

In FIG. 6C, six processing or reconstruction units 52₁, 52₂, . . . , 52₆ are configured to process two channels 42₁ and 42₂ with three pipeline stages 54₁, 54₂ and 54₃ each. The data from two coil elements are sent to two corresponding processing channels. The two images from each of the processing channels are summed up in the combining unit 44.

FIGS. 7A-C and 8 show two alternative implementations of the interconnections between the six processing or reconstruction units 52₁, 52₂, . . . , 52₆ of FIGS. 6A-C using a switch 60 or other hardware with similar functionality. The interconnections can be configured to realize the network topologies of FIGS. 6A-C. Although six processing units are shown by way of example, any number of processors could be used.

In FIG. 7A, a crossbar switch 60 is used to connect the six embedded processors 52₁, 52₂, . . . , 52₆ of FIG. 6A, which allows a static configuration of the connections 56 in hardware on a per scan basis. Each processor receives input data separately via the inputs I₁ through I₆. The processors 52₁, 52₂, . . . , 52₆ exchange images with each other via the crossbar 60. After completion of reconstruction, each processor sends an image via the outputs O₁ through O₆ to the combining unit 44. Alternatively, the image combination is performed partly or entirely on the processors themselves, as discussed above.

In FIG. 7B, a crossbar switch 60 is used to connect the six embedded processors 52₁, 52₂, . . . , 52₆ as shown in FIG. 6B. The processors 521, 523, and 525 are allocated to the pipeline stage 54₁ of channels 42₁, 42₂, and 42₃. The processors 52₁, 52₃, and 52₅ receive input data via the inputs I₁ through I₃. The processors 52₂, 52₄, and 52₆ are allocated to the pipeline stage 54₂ of channels 42₁, 42₂, and 42₃. The processors 52₂, 52₄, and 52₆ exchange images with each other via the crossbar 60. After completion of reconstruction, the processors 52₂, 52₄, and 52₆ send images via the outputs O₁ through O₃ to the combining unit 44.

In FIG. 7C, a crossbar switch 60 is used to connect the six embedded processors 52₁, 52₂, . . . , 52₆ as shown in FIG. 6C. The processors 52₁ and 52₄ are allocated to the pipeline stage 54₁ of channels 42₁ and 42₂. The processors 52₁ and 52₄ receive input data via the inputs I₁ and I₂. The processors 52₃ and 52₆ are allocated to the pipeline stage 54₃ of channels 42₁ and 42₂. The processors 52₃ and 52₆ exchange images with each other via the crossbar 60. After completion of the reconstruction, the processors 52₃ and 52₆ send images via the outputs O₁ and O₂ to the combining unit 44.

In FIG. 8, a switched fabric switch 60 is used to connect the six personal computers or workstations 52₁, 52₂, . . . , 52₆, each serving as one processing or reconstruction unit. The switch 60 permits a dynamic configuration of the connections 56 in software for each packet of data.

Thus, the systems shown in FIG. 7A-C and 8 can be configured for a first scan to have six processing channels with one pipeline stage each as per FIG. 7A; for a second scan to have three processing channels with two pipeline stages each as per FIG. 7B; and for a third scan to have two processing channels with three pipeline stages each as per FIG. 7C. Further, each processing or reconstruction unit need not be dedicated to a specific channel. Rather, one or more of the processing or reconstruction units can be shared between two or more channels.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. An MRI system comprising:
a means for creating and transmitting RF pulses into an examination region to excite and manipulate a spin system to be imaged;
a means for picking up an MR signal emitted from the examination region;
a means for demodulating the MR signal and converting the demodulated MR signal into digital data; and
a means for reconstructing images from the digital data, which includes:
a plurality of processing units, which include dynamically reconfigurable connections.

2. The MRI system as set forth in claim 1, wherein the plurality of processing units includes embedded processors.

3. The MRI system as set forth in claim 1, wherein the plurality of processing units includes one of personal computers and workstations.

4. The MRI system as set forth in claim 1, wherein the processing units are dynamically reconfigured utilizing a switched fabric, a crossbar or the like.

5. The MRI system as set forth in claim 1, wherein the means for picking up the MR signal includes a plurality of coil elements and the means for demodulating and converting the MR signal includes a plurality of RF receivers each operatively connected to an associated coil element, and further including:
a means for interconnecting the processing units to arrange the processing units into a plurality of independent parallel processing channels each channel being operatively connected with one or more RF receivers.

6. The MRI system as set forth in claim 5, wherein each of the independent parallel processing channels further include:
one or more pipeline stages.

7. The MRI system as set forth in claim 6, wherein each of the independent parallel processing channels further include:
a first pipeline stage to operate on the digital data in k-space;
one or more intermediate pipeline stages to transform the digital data from k-space to an image domain; and
a final pipeline stage to operate on the digital data in the image domain.

8. The MRI system as set forth in claim 6, further including:
a combining unit, operatively connected to the processing units allocated to a final pipeline stage, to manipulate outputs of each channel.

9. The MRI system as set forth in claim 8, wherein the combining unit weights the output of each channel and sums the weighted outputs.

10. The MRI system as set forth in claim 8, wherein an exchange of the data generated by the independent processing channels is restricted to an image domain and further includes:
one of the exchange of the data via the processing units allocated to the final pipeline stage and via the combining unit.

11. A method for processing an MR signal comprising:
creating and transmitting RF pulses into an examination region to excite and manipulate a spin system to be imaged;
picking up the MR signal emitted from the examination region;
demodulating the picked up MR signal and converting the demodulated MR signal into digital data; and
reconstructing images from the digital data via a plurality of processing units, which include dynamically reconfigurable connections.

12. The method as set forth in claim 11, further including:
dynamically reconfiguring the processing units connections to allocate the processing units to processing channels and pipeline stages on a per scan basis.

13. The method as set forth in claim 12, further including:
dynamically allotting the processing channels to RF receivers in use.

14. The method as set forth in claim 11, further including:
interconnecting the processing units to arrange the processing units into a plurality of independent parallel processing channels each channel being operatively connected with one or more RF receivers; and
reconstructing the images from the digital data via independent processing in each independent processing channel.

15. The method as set forth in claim 14, wherein the processing units in each independent parallel processing channel are arranged into a plurality of pipeline stages.

16. The method as set forth in claim 15, further including:
weighing an output of each processing channel; and
one of partial and complete combining of the weighed outputs.

17. The method as set forth in claim 16, wherein the combining is performed in a final pipeline stage and includes:
combining an image from a first channel with an image from an adjacent channel to form a first intermediate combined image, and combining an image from a channel n with an image from an adjacent channel to form a second intermediate combined image; and
combining each intermediate combined image with an image from another channel to generate new intermediate combined images until images from all channels have been combined into a resultant combined image.

18. The method as set forth in claim 17, further including:
distributing the resultant combined image to the processing units allocated to the final pipeline stage by consecutively forwarding the resultant combined image from the middle channel in direction of the last channel and simultaneously forwarding the resultant combined image in opposite directions from the middle channel in direction of the last channel via adjacent processing units.

19. The method as set forth in claim 16, wherein the combining is performed in a final pipeline stage and includes:
combining images from pairs of processing channels into intermediate combined images; and
combining pairs of the intermediate combined images until images from all channels have been combined into a resultant combined image.

20. The method as set forth in claim 19, further including:
distributing the resultant combined image to the processing units (52) allocated to the final pipeline stage (54ₘ) by consecutively forwarding the resultant combined image from the middle channel (42_{n/2}) to the last channel (42ₙ) and simultaneously forwarding the resultant combined image in opposite directions from the middle channel (42_{n/2}) to the last channel (42ₙ) via adjacent processing units.

21. The method as set forth in claim 14, further including:
mapping a forward processing of iterative reconstruction algorithms to the pipeline stages (54₁, 54₂, . . . , 54ₘ);
mapping a backward processing of the iterative reconstruction algorithms to the pipeline stages (54ₘ, 54ₘ₋₁, . . . , 54₁); and
simultaneously performing the forward and backward processing of different data sets, such that:
a first pipeline stage (54₁) operates on the digital data in k-space, and
a final pipeline stage (54ₘ) operates on the digital data in an image domain.

22. The method as set forth in claim 21, further including:
utilizing two separate independent parallel processing channels for the forward and backward processing of iterative reconstruction algorithms.
